(19) United States
(12) Patent Application Publication
(10) Pub. No.: US 2006/0204568 A1
(43) Pub. Date: Sep. 14, 2006

(54) Oral pharmaceutical preparation for proton pump antagonists

(76) Inventors: Rango Dietrich, Konstanz (DE); Bernd-Michael Haas, Reichenau (DE); Hartmut Ney, Konstanz (DE); Simone Hiltl, Konstanz (DE)

Correspondence Address: NATH & ASSOCIATES PLLC, 112 South West Street, Alexandria, VA, 22314, US

(21) Appl. No.: 10/551,943

(22) PCT Filed: Apr. 8, 2004
(86) PCT No.: PCT/EP04/50493

§ 371 Date: Nov. 17, 2005

(30) Foreign Application Priority Data
Apr. 11, 2003 (DE) 10317023.5
Apr. 11, 2003 (EP) 03008453.7

(51) Int. Cl. A61K 9/20; A61K 31/4439

(52) U.S. Cl. 424/464; 514/338

(57) ABSTRACT

The invention relates to novel dosage forms for proton pump antagonists.

### TECHNICAL FIELD

The present invention relates to oral pharmaceutical preparations in multiparticulate form or in tablet form for proton pump antagonists.

### STATE OF THE ART

Irreversible proton pump inhibitors (H⁺/K⁺-ATPase inhibitors, PPIs), especially pyridin-2-ylmethyl-sulphinyl-1H-benzimidazoles as disclosed for example in EP-A-0 005 129, EP-A-0 166 287, EP-A-0 174 726 and EP-A-0 268 956, have, by reason of their H⁺/K⁺-ATPase inhibiting effect, importance in the therapy of diseases derived from increased gastric acid secretion. Irreversible proton pump inhibitors are substances which bind covalently, and thus irreversibly, to the enzyme responsible for acid secretion in the stomach, the H⁺/K⁺⁻ ATPase [description of the mechanism of action for example in Wurst et al., The Yale Journal of Biology and Medicine 69, (1996), 233-243]. Examples of commercially available active ingredients from this group are 5-methoxy-2-[(4-methoxy-3,5-dimethyl-2-pyridinyl)methylsulphinyl]-1H-benzimidazole (INN: omeprazole), 5-difluoromethoxy-2-[(3,4-dimethoxy-2-pyridinyl)methylsulphinyl]-1H-benzimidazole (INN: pantoprazole), 2-[3-methyl 2,2,2-trifluoroethoxy)-2-pyridinyl)methylsulphinyl]-1H-benzimidazole (INN: lansoprazole) and 2-[[4-(3-methoxypropoxy)-3-methylpyridin-2-yl]methylsulphinyl]-1H-benzimidazole (INN: rabeprazole)

Because of their great tendency to decompose in a neutral and, in particular, acidic environment, with highly coloured decomposition products also being formed, it is necessary for oral preparations to protect the irreversible proton pump inhibitors from the action of acids. With the highly acid-labile pyridin-2-yl-methylsulphinyl-1H-benzimidazoles it is additionally necessary for them to be processed in the tablet core or in pellets in the form of their alkaline salts, for example as sodium salts, or together with alkaline substances. Since the materials suitable for enteric coatings have free carboxyl groups, the problem arises that the enteric coating is partly or even completely dissolved from the inside, because of the alkaline milieu in the interior, and the free carboxyl groups promote decomposition of the active ingredients, it is therefore necessary to provide a sealing intermediate layer (subcoating) between the enteric coating and the alkaline tablet core or pellet. EP-A-0 244 380 proposes coating cores which contain the active ingredient together with alkaline compounds or as alkaline salt with at least one layer which is soluble in water or disintegrates rapidly in water and is composed of nonacidic inert pharmaceutically acceptable substances, before the enteric layer is applied. The intermediate layer or intermediate layers act as pH-buffering zones in which the hydrogen ions diffusing in from the outside are able to react with the hydroxyl ions diffusing out of the alkaline core. In order to increase the buffer capacity of the intermediate layer, it is proposed to incorporate buffer substances into the intermediate layer(s). It is possible by this process in practice to obtain reasonably stable preparations. However, relatively thick intermediate layers are necessary in order to prevent the unsightly discolourations occurring even if there is only slight decomposition. In addition, considerable effort must be made to avoid traces of moisture during production.

Besides the so-called irreversible proton pump inhibitors which, as mentioned at the outset, essentially have a common basic chemical structure (they are pyridinylmethylsulphinylbenzimidazoles), there are so-called reversible H⁺/K⁺-ATPase inhibitors which have different basic chemical structures and which—as indicated by the name—reversibly bind to the enzyme responsible for gastric acid secretion and are therefore also called proton pump antagonists or APAs (=acid pump antagonists) [description of the mechanism of action for example in Wurst et al, The Yale Journal of Biology and Medicine 69 (1996), 233-243). Reversible proton pump inhibitors are disclosed for example in the documents DE-A 3917232, EP-A-0399267, EP-A-0387821, JP-A-3031280, JP-A-2270873, EP-A-0308917, EP-A-0268989, EP-A-0228006, EP-A-0204285, EP-A-0165545, EP-A-0125756, EP-A-0120589, EP-A-0509974, DE-A 3622036, EP-A-0537532, EP-A 535529, JP-A-3284686, JP-A-3284622, U.S. Pat. No. 4,833,149, EP-A-0261912, WO-A-114677, WO-A-9315055, WO-A-9315071, WO-A-9315056, WO-A-9312090, WO-A-9212969, WO-A-9118887, EP-A-0393926, EP-A-0307078, U.S. Pat. No. 5,041,442, EP-A-0266890, WO-A-9414795, EP-A-0264883, EP-A-0033094, EP-A-0259174, EP-A-0330485, WO-A-8900570, EP-A-0368158, WO-A-9117164, WO-A-9206979, WO-A-9312090, WO-A-9308190, WO-A-9418199, DE-A 3011490, U.S. Pat. No. 4,464,372 EP-A-0068378 and WO-A-9424130.

EP 0841904 B1 describes an oral pharmaceutical composition with delayed release of reversible proton pump inhibitors in combination with antimicrobial active ingredients for the treatment of a disease caused by helicobacter.

WO-A-9527714 is related to substituted tricyclic imidazo[1,2-a]pyridines which reversibly inhibit exogenously or endogenously stimulated gastric acid secretion. On page 38 an example for a tablet formulation is disclosed.

WO-A-0245693 discloses new preparations for an active ingredient, wherein the active ingredient is present essentially uniformly dispersed in an excipient matrix composed of one or more excipients selected from the group of fatty alcohol, triglyceride, partial glyceride and fatty acid ester. It is mentioned that the matrix is inter alia suitable for active ingredients from the class of substances known as reversible propion pump inhibitors or APAs (acid pump antagonists). Rapidly disintegrating tablets based on these preparations are mentioned.

### DESCRIPTION OF THE INVENTION

It has surprisingly been found that particularly stable oral dosage forms are obtained for proton pump antagonists (APA) when the active ingredient is stabilized in the dosage form by basic excipients.

One aspect of the invention is therefore a stable oral dosage form for reversible proton pump inhibitors comprising an effective amount of a proton pump antagonist (APA) together with excipients, where the proton pump antagonist is stabilized in the dosage form by one or more basic excipients.

It has also surprisingly been found that therapeutic advantages can be achieved for oral administration through the administration of proton pump antagonists (APAs) by means of a rapidly disintegrating dosage form, preferably with an immediate release of the active ingredient. In particular, a faster onset of action and a faster elimination of pain are observed in the therapy of diseases derived from increased gastric acid secretion.

A further aspect of the invention is therefore also a rapidly disintegrating dosage form comprising an effective amount of a proton pump antagonist (APA) together with excipients which, on oral intake of the dosage form, bring about rapid disintegration of the dosage form, and, where appropriate, further excipients. Preferably the dosage form shows an immediate release of the active ingredient.

Irreversible proton pump inhibitors (H⁺/K⁺-ATPase inhibitors, PPIs) are according to the invention substances which are able to bind covalently, and thus irreversibly, to the enzyme responsible for acid secretion in the stomach, H⁺/K⁺-ATPase [description of the possible mechanism of action for example in Wurst et al., The Yale Journal of Biology and Medicine 69, 3, 1996, 233-243]. By this are meant in particular pyridin-2-yl-methylsulphinyl-1H-benzimidazoles as disclosed for example in EP-AD 005 129, EP-A-0 166 287, EP-A0 174 726 and EP-A0 268 956. Examples which may be mentioned are 5-methoxy-2[(4-methoxy-3,1-dimethyl-2-pyridinyl)methylsulphinyl-1H-benzimidazole (INN: omeprazole), 5-difluoromethoxy-2-[(3,4-dimethoxy-2-pyridinyl)methylsulphinyl]-1H-benzimidazole (INN: pantoprazole), 2-[3-methyl-4-(2,2,2-trifluoroethoxy)-2-pyridinyl)methylsulphinyl]-1-benzimidazole (INN: lansoprazole) and 2-{[4-(3-methoxypropoxy)₃-methylpyridin-2-yl]methylsulphinyl}-1H-benzimidazole (INN: rabeprazole).

Proton pump antagonists, also called according to the invention reversible proton pump inhibitors or APA (acid pump antagonists), are for the purposes of the present invention those active ingredients able to bind reversibly to the enzyme responsible for gastric acid secretion H⁺/K⁺-ATPase [description of the possible mechanism of action of the APAs for example in Wurst et al, The Yale Journal of Biology and Medicine 69, 3, 1996, 233-243]. The term proton pump antagonists includes according to the invention not only the active ingredient as such but also the pharmacologically acceptable salts and solvates (especially hydrates) etc. Examples of proton pump antagonists are mentioned in the following documents: EP 33094, EP 204285, EP 228006, EP 233760, EP 259174. EP 266326, EP 266890, EP 270091, EP 307078, EP 308917, EP 330485, U.S. Pat. No. 4,728,658, U.S. Pat. No. 5,362,743, WO 9212969, WO 9414795, WO 9418199, WO 9429274, WO 9510518, WO 9527714, WO 9603405, WO 9604251, WO 9605177, WO 9703074, WO 9703076, WO 9747603, WO 9837080, WO 9842707, WO 9843968, WO 9854188, WO 9909029. WO 9928322, WO 9950237, WO 9951584, WO 9955705, WO 9955708, WO 001696, WO 0010999, WO 0011000, WO 0017200, WO 0026217, WO 0029403, WO 0063211, WO 0077003, WO 0158901, WO 0172754, WO 0172755, WO 0172756, WO 0172757, WO 02034749, WO 02060440, WO 02060441 and WO 02060442.

Examples of proton pump antagonists which may be mentioned by means of their INNs or their code designation are the compounds: AG-2000 (EP 233760), AU-461 (WO 9909029), BY112 (WO 9842707), soraprazan (BY359) (WO 0017200), CP-113411 (U.S. Pat. No. 5,362,743), DBM-819 (WO 0001696), KR-60436 (WO 9909029), pumaprazole (WO 9418199), SKF-46067 (EP 259174), SKF-96356 (EP 307078), SKF-97574 (EP 330485), T-0 (EP 270091), T-776 (EP 270091), WY-27198 (U.S. Pat. No. 4,728,658), YH-1885 (WO 9605177), YJA-20379-8 (WO 9703074), YM-19020 (EP 266890) and 2,3-dimethyl-8-(2-ethyl-6-methylbenzylamino)imidazo[1,2-a]pyridine-6-carboxamide (WO 02060440).

Particularly worthy of mention in this connection are the compounds AU-461, soraprazan (BYK61359), DBM-819, KR-40436, T-330, YH-1885, YJA-20379-8 and 2,3-dimethyl-8-(2-ethyl-6-methylbenzylamino)imidazol-1,2-a]pyridine-carboxamide.

A group of APAs which is of particular interest according to the invention is described and claimed in the patent applications WO 9842707, WO 9854188, WO 0017200, WO 0026217, WO 0063211, WO 0172754, WO 0172755, WO 0172756, WO 0172757, WO 02034749, WO-03014120, WO-03016310, WO03014123, WO03068774 and WO03091253.

Examples of APAs which may be mentioned in connection with the invention are the following compounds:
(7S,8R,9R)-2,3-dimethyl-7,8-dihydroxy-9-phenyl-7,8,9,10-tetrahydroimidazo[1,2-h][1,7]naphthyridine,
(7R,8R,9R)₄-hydroxymethyl-7,8-dihydroxy-2-methyl-9-phenyl-7,8,9,10-tetrahydroimidazo[1,2-h][1,7]naphthyridine,
(7S,8R,9R)-7,8-isopropylidenedioxy-2,3-dimethyl-phenyl-7,8,9,10-tetrahydroimidazo[1,2-h][1,7]naphthyridine,
7,8-dihydroxy-9-phenyl-2,3-dimethyl-7H-8,9-dihydropyrano[2,3-c]imidazo[1,2-a]pyridine,
(7R,8R,9R)-2,3-dimethyl-8-hydroxy-7-methoxyphenyl-7,8,9,10-tetrahydroimidazo[1,2-h]1,7]naphthyridine,
(7S,8S,9S)-2,3-dimethyl-hydroxy-7-methoxy-phenyl-7,8,9,10-tetrahydroimidazo[1,2-h][1,7]naphthyridine,
(7S,8R,9R)-2,3-dimethyl-hydroxy-7-methoxy-phenyl-7,8,9,1-tetrahydroimidazo[1,2-h][1,7]naphthyridine,
(7R,8S,9S)-2,3-dimethyl-hydroxy-7-methoxy-phenyl-7,8,9,10-tetrahydroimidazo[1,2-h][1,7]naphthyridine,
(7R,8R,9R)-2,3-dimethyl-7-ethoxy-8-hydroxy-9-phenyl-7,8,9,10-tetrahydroimidazo[1,2-h][1,7]naphthyridine,
(7S,8R,9R)-2,3-dimethyl-7-ethoxy-8-hydroxy-9-phenyl-7,8,9,10-tetrahydroimidazo[1,2-h][1,7]naphthyridine,
(7R,8R,9R)-2,3-dimethyl-hydroxy-7-(2-methoxyethoxy)-9-phenyl-7,8,9,10-tetrahydroimidazo[1,2-h][1,7]naphthyridine,
(7S,8S,9S)-2,3-dimethyl-8-hydroxy-7-(2-methoxyethoxy)-9-phenyl-7,8,9,10-tetrahydroimidazo[1,2-h][1,7]naphthyridine,
(7S,8R,9R)-2,3-dimethyl-8-hydroxy-7-(2-methoxyethoxy)-9-phenyl-7,8,9,10-tetrahydroimidazo[1,2-h][1,7]naphthyridine,
(7R,8S,9S)-2,3-dimethyl-8-hydroxy-7-(2-methoxyethoxy)-9-phenyl-7,8,9,10-tetrahydroimidazo[1,2-h][1,7]-naphthyridine,
(7S,8R,9R)-2,3-dimethyl-8-hydroxy-9-phenyl-7,8,9,10-tetrahydroimidazo[1,2-h][1,7]naphthyridine,
(7R,8R,9R)-2,3-dimethyl-7,8-dimethoxy-9-phenyl-7,8,9,10-tetrahydroimidazo[1,2-h][1,7]naphthyridine,
(7R,8R,9R)-2,3-dimethyl-8-hydroxy-7-(2-methylthioethyloxy)-phenyl-7,8,9,10-tetrahydroimidazo[1,2-h][1,7]naphthyridine,
(7S,8R,9R)-2,3-dimethyl-hydroxy-7-(2-methylthioethyloxy)-9-phenyl-7,8,9,10-tetrahydroimidazo[1,2-h][1,7]naphthyridine,
(7R,8R,9R)-2,3-dimethyl-hydroxy-7-(2-methylsulphinylethoxy-9-phenyl-7,8,9,10-tetrahydroimidazo[1,2-h][1,7]naphthyridine,
(7S,8R,9R)-2,3-dimethyl-8-hydroxy-7-(2-methylsulphinylethoxy-9-phenyl-7,8,9,10-tetrahydroimidazo[1,2-h][1,7]naphthyridine,
(7R,8R,9R)-2,3-dimethyl-8-hydroxy-7-(ethylthio)-9-phenyl-7,8,9,10-tetrahydroimidazo[1,2-h][1,7]naphthyridine,
(7S,8R,9R)-2,3-dimethyl-hydroxy-7-(ethylthio)-9-phenyl-7,8,9,10-tetrahydroimidazo[1,2-h][1,7]naphthyridine,
(7R,8R,9R)-2,3-dimethyl-9-hydroxy-7-(2,2,2-trifluoroethoxy)-9-phenyl-7,8,9,10-tetrahydroimidazo[1,2-h][1,7]naphthyridine,
(7S,8R,9R)-2,3-dimethyl-9-hydroxy-7-(2,2,2-trifluoroethoxy)-9-phenyl-7,8,9,10-tetrahydroimidazo[1,2-h)-[1,7]naphthyridine,
(7S,8R,9R)-8-acetoxy-7-(2-methoxyethoxy)-2,3-dimethyl-phenyl-7,8,9,10-tetrahydroimidazo[1,2-h]-[1,7]naphthyridine,
(7R,8R,9R)-acetoxy-7-(2-methoxyethoxy)-2,3-dimethyl-9-phenyl-7,8,9,10-tetrahydroimidazo[1,2-h]-[1,7]naphthyridine,
(7R,8R,9R)-8-acetoxy-7-methoxy-2,3-dimethyl-9-phenyl-7,8,9,10-tetrahydroimidazo[1,2-h][1,7]naphthyridine,
(7R,8R,9R)-8-acetoxy-7-ethoxy-2,3-dimethyl-9-phenyl-7,8,9,10-tetrahydroimidazo[1,2-h][1,7]naphthyridine,
(7R,8R,9R)-7-(2-methoxyethoxy-2,3-dimethyl-9-phenyl-8-propionyloxy-7,8,9,10-tetrahydroimidazo[1,2-h][1,7]naphthyridine,
(7R,8R,9R)-8-benzoyloxy-7-(2-methoxyethoxy)-2,3-methylphenyl-7,8,9,10-tetrahydroimidazo[1,2-h][1,7]naphthyridine,
7S,8R,9R)-8-benzoyloxy-7-(2-methoxyethoxy)-2,3-dimethylphenyl-7,8,9,10-tetrahydroimidazo[1,2-h][1,7]naphthyridine,
(7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7S,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-7-methoxy-2,3-dimethyl-9-phenyl-7,8,9,10-tetrahydroimidazo[1,2-h][1,7]naphthyridine,
(7S,8R,9R)-8-benzoyloxy-7-methoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7S,8R,9R)-7-methoxy-2,3-dimethyl-8-(4-nitrobenzoyloxy-9-phenyl-7,8,9,10-tetrahydroimidazo[1,2-h]-[1,7]naphthyridine,
(7R,8R,9R)-7-methoxy-2,3-dimethyl-8-(3-nitrobenzoyloxy)-9-phenyl-7,8,9,10-tetrahydroimidazo[1,2-h]-[1,7]naphthyridine,
(7S,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-(N,N-dimethylaminomethylcarbonyloxy)-9-phenyl-7,8,9,10-tetrahydroimidazo[1,2-h][1,7]naphthyridine,
(7S,8R,9R)-7-(2-Methoxyethoxy)-2,3-dimethyl-8-(N,N-dimethylaminomethylcarbonyloxy)-9-phenyl-7,89,10-tetrahydroimidazo[1,2-h][1,7]naphthyridine,
(7S,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-7-(2-methoxyethoxy)-(N,N-diethylaminocarbonyloxy)-2,3-dimethyl-9-phenyl-7,8,9,10-tetrahydroimidazo[1,2-h][1,7]naphthyridine,
(7R,8R,9R)-8-ethylaminocarbonyloxy-7-(2-methoxyethoxy)-2,3-dimethyl-9-phenyl-7,8,9,10-tetrahydroimidazo[1,2-h[1,7]naphthyridine,
(7R,8R,9R)-8-benzoyloxy-2,3-dimethyl-7-(2-methoxyethoxy)-9-phenyl-7H-8,9-dihydropyrano[2,3-c]imidazo[1,2-a]pyridine,
(7S,8R,9R)-8-benzoyloxy-2,3-dimethyl-7-(2-methoxyethoxy)-9-phenyl-7H-8,9-dihydropyrano[2,3c]imidazo[1,2-a]pyridine,
(7R,8R,9R)-8[4-(methoxycarbonyl)benzoyloxy)-2,3-dimethyl-7-(2-methoxyethoxy)-9-phenyl-7H-8,9-dihydropyrano[2,3c]imidazol-1,2-]pyridine,
(7S,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-2.3 dimethyl-7-methoxy-8-methoxyacetyloxy-9-phenyl-7.8.9.10-tetrahydroimidazo[1.2-h][1.7]naphthyridine,
(7R,8R,9R)-8-(N,N-diethylaminocarbonyloxy-2.3-dimethyl-7-methoxy-9-phenyl-7.8.9.10-tetrahydroimidazo[1.2-h][1.7]naphthyridine,
(7S,8R,9R)-8-(N,N-diethylaminocarbonyloxy)-2.3-dimethyl-7-methoxy-9-phenyl-7.8.9.10-tetrahydroimidazo[1.2-h]1.7]naphthyridine,
(7R,8R,9R)-7-methoxy-8-methoxycarbonyloxy-2.3-dimethyl-9-phenyl-7.8.9.10-tetrahydroimidazo[1.2-h]-[1.7]naphthyridine,
(7S,8R,9R)-7-methoxy-8-methoxycarbonyloxy-2.3-dimethyl-phenyl-7.8.9.10-tetrahydroimidazo[1.2-h][1.7]naphthyridine,
(7R,8R,9R)-2.3-dimethyl-8-formyloxy-7-methoxy-9-phenyl-7.8.9.10-tetrahydroimidazo[1.2-h][1.7]naphthyridine,
(7S,8R,9R)-2.3-dimethyl-8-formyloxy-7-methoxy-9-phenyl-7.8.9.10-tetrahydroimidazo[1.2-h][1.7]naphthyridine,
(7R,8R,9R)-benzoyloxy-2.3-dimethyl-7-methoxy-9-phenyl-7.8.9.10-tetrahydroimidazo[1.2-h][1.7]naphthyridine,
(7R,8S,9R)-2,3,8-trimethyl-7,8-dihydroxyphenyl-7,8,9,10-tetrahydroimidazo[1,2h][1,7]naphthyridine,
(7S,8S,9R)-2,3-dimethyl-8-benzyl-7,8-dihydroxy-phenyl-7,8,9,10-tetrahydroimidazo[1,2-h][1,7]naphthyridine,
(7R,8S,9R)-2,3,8-trimethyl-7,8,0,0-isopropylidene-9-phenyl-7,8,9,10-tetrahydroimidazo[1,2-h][1,7]naphthyridine,
(7S,8S,9R)-2,3-trimethyl-7-(2-methoxyethoxy)-8-hydroxy-9-phenyl-7,8,9,10-tetrahydroimidazo[1,2-h][1,7]naphthyridine,
(7S,8S,9R)-2,3,8-trimethyl-7-methoxy-8-hydroxy-9-phenyl-7,8,9,10-tetrahydroimidazo[1,2-h][1,7]naphthyridine,
(7R,8R,9R)-2,3,7-trimethyl-7,8-dihydroxy-9-phenyl-7,8,9,10-tetrahydroimidazo[1,2-h][1,7]naphthyridine,
(7R,8R,9R)-2,3,7-trimethyl-7,8-[1,3]dioxolo-9-phenyl-7,8,9,10-tetrahydroimidazo[1,2-h][1,7]naphthyridine,
(8S,9R)-2,3-dimethyl-8-hydroxy-7-methylidene-9-phenyl-7,8,9,10-tetrahydroimidazo[1,2-h][1,7]naphthyridine,
(7S,8R,9R)-2,3,7-trimethyl-7,8-dihydroxy-9-phenyl-7H-8,9-dihydropyrano[2,3-c]imidazo[1,2-a]pyridine,
(7R,8R,9R)-2,3,7-trimethyl-7,8-dihydroxy-9-phenyl-7H-8,9-dihydropyrano[2,3-c]imidazo[1,2-a]pyridine
(7S,8R,9R)-2,3-dimethyl-7,8-dihydroxy-7,9-diphenyl-7H-8,9-dihydropyrano[2,3-c]imidazol-1,2-a]pyridine.
(7S,8R,9R)-2,3-dimethyl-7-(2′,2′-dimethylvinyl)-7,8-dihydroxy-9-phenyl-7H-8,9-dihydropyrano[2,3-c]imidazo[1,2-a]pyridine,
(7R,8R,9R)-2,3-dimethyl-7,8-O-isopropylidene-phenyl-7-vinyl-7H-8,9-dihydropyrano[2,3-c]imidazo[1,2-a]pyridine,
(7R,8R,9R)-2,3-dimethyl-8-hydroxy-7-(2-methoxyethoxy)-9-phenyl-7H-8,9-dihydropyrano[2,3-c]imidazo[1,2-a]pyridine,
(7S,8R,9R)-2,3-dimethyl-8-hydroxy-7-(2-methoxyethoxy)-9-phenyl-7H-8,9-dihydropyrano[2,3-c]imidazo[1,2-a]pyridine,
(7R,8R,9R)-2,3-dimethyl-8-hydroxy-7-ethoxy-9-phenyl-7H-8,9-dihydropyrano[2,3-c]imidazo[1,2-a]pyridine,
(7S,8R,9R)-2,3-dimethyl-8-hydroxy-7-ethoxy-9-phenyl-7H-8,9-dihydropyrano[2,3-c]imidazo[1,2-c]imidazo[1,2-a]pyridine,
(7R,8R,9R)-2,3-dimethyl-8-hydroxy-7-(2-methoxypropoxy)-9-phenyl-7H-8,9-dihydropyrano[2,3-c]imidazo[1,2-a]pyridine,
(7S,8R,9R)-2,3-dimethyl-8-hydroxy-7-(2-methoxypropoxy)-9-phenyl-7H-8,9-dihydropyrano[2,3-c]imidazo[1,2-a]pyridine,
(7R,8R,9R)-2,3-dimethyl-8-hydroxy-7-(2-propoxy)-9-phenyl-7H-8,9-dihydropyrano[2,3-c]imidazo[1,2-a]pyrimidine,
(7S,8R,9R)-2,3-dimethyl-8-hydroxy-7-(2-propoxy)-9-phenyl-7H-8,9-dihydropyrano[2,3-c]imidazo[1,2-a]pyridine,
(7R,8R,9R)-2,3-dimethyl-8-hydroxy-7-butoxy-9-phenyl-7H-8,9-dihydropyrano[2,3c]imidazo[1,2-a]pyridine,
(7S,8R,9R)-2,3-dimethyl-8-hydroxy-7-butoxy-9-phenyl-7H-8,9-dihydropyrano[2,3-c]imidazo[1,2-a]pyridine,
(7S,8R,9R)-7,8-dihydroxy-6-methoxymethyl-2,3-dimethyl-9-phenyl-7,8,9,10-tetrahydroimidazo[1,2-h][1,7]-naphthyridine,
(7R,8R,9R)-7,8-dihydroxy-6-methoxymethyl-2,3-dimethyl-9-phenyl-7,8,9,10-tetrahydroimidazo[1,2-h][1,7]naphthyridine,
(7S,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-methoxy-6-methoxymethyl-2,3-dimethyl-phenyl-7,8,9,10-tetrahydroimidazo[1,2-h][1,7]naphthyridine,
(7R,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7S,8R,9R)-8-Hydroxy-7-(2-methoxyethoxy)methoxymethyl-2,3-dimethyl-9-phenyl-7,8,9,10-tetrahydroimidazo[1,2-h][1,7]naphthyridine,
(7R,8R,9R)-8-hydroxy-7-ethoxy-6-methoxymethyl-2,3-dimethyl-9-phenyl-7,8,9,10-tetrahydro-imidazo[1,2-h][1,7]naphthyridine,
(7S,8R,9R)-8-hydroxy-7-ethoxy-6-methoxymethyl-2,3-dimethyl-9-phenyl-7,8,9,10-tetrahydro-imidazo[1,2-h][1,7]naphthyridine,
7,8-dihydroxy-2,3-dimethyl-9-(3-thienyl)-7,8,9,10-tetrahydroimidazo[1,2-h][1,7]naphthyridine,
7-hydroxy-2,3-dimethyl-9-(3-thienyl)-7,8,9,10-tetrahydroimidazo[1,2-h][1,7]naphthyridine,
9-(3-furyl)-7-hydroxy-2,3-dimethyl-7,8,9,10-tetrahydroimidazo[1,2-h][1,2-h][1,7]napthyridine,
(7R,8R,9R)-8-hydroxy-7-[2-(2-methoxyethoxy)ethoxy]2,3-dimethyl-9-phenyl-7,8,9,10-tetrahydroimidazo[1,2-h][1,7]naphthyridine,
(7S,8R,9R)-8-hydroxy-7-[2-(2-methoxyethoxy)ethoxy]2,3-dimethyl-9-phenyl-7,8,9,10-tetrahydroimidazo[1,2-h][1,7]naphthyridine,
(7R,8R,9R)-7,8-dihydroxy-2-methyl-9-phenyl-7,8,9,10-tetrahydroimidazo[1,2-h][1,7-h][1,7]naphthyridine,
(7S,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-3-bromo-8-hydroxy-7-(2-methoxyethoxy)-2-methyl-9-phenyl-7.8.9.10-tetrahydroimidazo[1.2-h][1.7]napthyridine,
(7R,8R,9R)-3-chloro-8-hydroxy-7-(2-methoxyethoxy)-2-methyl-9-phenyl-7.8.9.10-tetrahydroimidazo[1.2-h][1.7]napthyridine,
(7R,8R,9R)-3-bromo-7-hydroxy-8-(2-methoxyethoxy)-2-methyl-9-phenyl-7.8.9.10-tetrahydroimidazo[1.2-h][1.7]naphthyridine,
(7R,8R,9R)-3-chloro-8-hydroxy-7-(2-methoxyethoxy)-2-methyl-9-phenyl-7H-8,9-dihydro-pyrano[2,3-c]imidazo[1,2-a]pyridine,
(7R,8R,9R)-8-hydroxy-7-(2-methoxyethoxy)-2-methyl-9-phenyl-7H-8,9-dihydropyrano[2,3-c]imidazo[1,2-a]pyridine,
(7R,8R,9R)-7,8-dihydroxy-2-methyl-9-phenyl-7H-8,9-dihydropyrano[2,3-c]imidazo[1,2-a]pyridine,
(7S,8R,9R)-7,8-dihydroxy-2-methyl-9-phenyl-7.8.9.10-tetrahydroimidazo[1.2-h][1.7]naphthyridine,
(7R,8R,9R)-8-hydroxy-7-methoxy-2-methyl-9-phenyl-7.8.9.10-tetrahydroimidazo[1.2-h][1.7]napthyridine
(7S,8R,9R)-8-hydroxy-7-methoxy-2-methyl-9-phenyl-7.8.9.10-tetrahydroimidazo[1.2-h][1.7]-napthyridine,
(7R,8R,9R)-3-hydroxymethyl-8-hydroxy-7-(2-methoxyethoxy)-2-methyl-9-phenyl-7.8.9.10-tetrahydroimidazo[1.2-h][1.7]naphthyridine,
(7R,8R,9R)-3-hydroxymethyl-8-hydroxy-8-(2-hydroxyethoxy)-2-methyl-9-phenyl-7.8.9.10-tetrahydroimidazo[1,2-h][1.7]naphthyridine,
(7R,8R,9R)-2,3-dimethyl-8-hydroxy-7-(2-hydroxyethoxy)-9-phenyl-7.8.9.1-tetrahydroimidazo[1.2-h][1.7]naphthyridine,
(7R,8R,9R)-3,9-diphenyl-8-hydroxy-7-(2-methoxyethoxy)-2-methyl-7.8.9.10-tetrahydroimidazo[1.2-h][1.7]naphthyridine,
(7R,8R,9R)-7,8-dihydroxy-2-methoxymethyl-4-methyl-9-phenyl-7,8,9,10-tetrahydroimidazo[1,2-h][1,7]naphthyridine,
(7S,8R,9R)-8-hydroxy-7-(2-methoxyethoxy)-2-methoxymethyl-3-methyl-9-phenyl-7,8,9,10-tetrahydroimidazo[1,2-h][1.7]naphthyridine,
(7R,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7S,8R,9R)-7-ethoxy-8-hydroxy-2-methoxymethyl-3-methyl-9-phenyl-7,8,9,10-tetrahydroimidazo[1,2-h][1.7]naphthyridine,
(7R,8R,9R)-7-ethoxy-8-hydroxy-2-methoxymethyl-4-ethyl-phenyl-7,8,9,10-tetrahydroimidazo[1,2-h][1.7]naphthyridine,
(8S)-2,3-Dimethyl-8-phenyl-7,8-dihydro-6H-9oxa-1,3a-diaza-cyclopenta[a]naphthalene-5-carboxylic acid dimethylamid,
8-[(1S,2S)-2,3-dihydro-2-hydroxy-1-indenyloxy-6-(N,N-dimethylaminocarbonyl)-2,3-dimethyl-imidazo[1,2-a]pyridine,
6-(N,N-Dimethylaminocarbonyl)-4-(2,6-dimethyl-benzylamino)-1,2-dimethyl-1H-benzimidazole, and the pharmacologically suitable salts of these compounds.

An example of a preferred proton pump antagonist which may be mentioned is the compound (7R,8R,9R)-2,3-dimethyl-hydroxy-7-(2-methoxyethoxy)-9-phenyl-7,8,9,10-tetrahydroimidazo[1,2-h][1,7]naphthyridine (INN: soraprazan).

The proton pump antagonists may in this connection be present as such or in the form of their salts and/or solvates (e.g. hydrates) etc. Most reversible proton pump inhibitors are basic compounds. Particularly suitable salts are all acid addition salts. Particular mention may be made of the pharmacologically acceptable salts of the inorganic and organic acids normally used in pharmaceutical technology. Suitable as such are water-soluble and water-soluble acid addition salts with acids such as, for example, hydrochloric acid, hydrobromic acid, phosphoric add, nitric acid, sulphuric acid, acetic acid, citric add, D-gluconic acid, benzoic add, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 3-hydroxy-2-naphthoic acid, the acids being employed in the preparation of the salts, depending on whether the acid is mono- or polybasic and on which salt is desired—in the equimolar ratio of amounts or one differing therefrom.

The dosage form according to the invention is preferably a solid dosage form in multiparticulate form (multiple unit dosage form) or in tablet form for oral administration. Examples which may be mentioned are, in particular, tablets, coated tablets, coloured tablets, pellets, microtablets in capsules or granules in capsules. A preferred embodiment comprises a tablet or pellets with a film coating or a coloured tablet. A film coating in the case of the film coating preferably does not impede rapid disintegration of the dosage form. In the case of proton pump antagnoists which are photosensitive, tablets or pellets according to the invention contain a film coating which protects the active ingredient from photodecomposition. The film coating in this case is particularly preferably coloured. In another embodiment, a colouring agent is included in the process to produce the tablet cores or pellets, and the solid dosage form is coloured. The dosage forms according to the invention preferably do not show, in contrast to the dosage forms described in EP-0841904-B1, delayed release but show immediate release of the active ingredient. Preference is therefore given according to the invention to a rapidly disintegrating dosage form with immediate release of the active ingredient (immediate release solid oral dosage form). The dosage form preferably has a maximum disintegration time in water (at 37° C.) of 15 minutes, 10 minutes, 5 minutes, 4 minutes or 3 minutes. Preferably the disintegration time is not exceeding 15 minutes, 10 minutes, 5 minutes, 4 minutes or 3 minutes in water with a temperature of 15 to 25° C. (The disintegration time of the tablet can be determined according to standard procedures disclosed in pharmacopoeia monographs, preferably according to the European Pharmacopoeia 4^{th} edition). The dosage form preferably has a release of active ingredient of greater than or equal to 60% after 15 minutes in 0.1 N hydrochloric acid, particularly preferable greater than or equal to 75% after 15 minutes in 0.1 N hydrochloric acid, more particularly preferable greater than or equal to 80% after 15 minutes in 0.1 N hydrochloric acid and even more particularly preferable greater than or equal to 85% after 15 minutes in 0.1 N hydrochloric acid. In a preferred embodiment the dosage form has a release of active ingredient of greater than or equal to 90% after 15 minutes and preferably a release of active ingredient of greater than or equal to 95% after 30 minutes (label claim).

In one embodiment of the invention, the rapidly disintegrating dosage form according to the invention is a dosage form displaying the properties according to the pharmacopoeia monographs in the European Pharmacopoeia 4^{th} edition “Tablets for preparing a suspension to be taken (dispersible tablet)” or “Tablets for preparing a solution to be taken”. Particular preference is moreover given according to the invention to solid, rapidly disintegrating dosage forms which show a maximum disintegration time of 10 minutes, 5 minutes, 4 minutes or 3 minutes under the test conditions described in European Pharmacopoeia 4^{th} edition for “dispersible tablets” (in cold water at a temperature of 15 to 25° C.) and leave no residues on a screen of mesh size 710 μm.

In a preferred embodiment the dosage form according to the invention is a rapidly disintegrating dosage form which shows a disintegration time determined in water at 37° C. of not more than 5 min and a dissolution (release of active ingredient) greater than or equal to 85% after 15 minutes in 0.1 N hydrochloric acid.

The dosage forms according to the invention are distinguished by rapid disintegration, rapid release of active ingredient and an optimal action profile (e.g. a rapid onset of action) in the therapy of diseases derived from increased gastric add secretion. There is furthermore observed to be an improved stability of the proton pump antagonists in dosage forms according to the invention containing a basic excipient.

Basic excipients which are suitable according to the invention and which can be employed in the dosage forms according to the invention to stabilize the proton pump antagonists are substances which have a basic reaction and are pharmacologically acceptable and able to stabilize the proton pump antagonists in the dosage form. These are, in particular, compounds selected from the group of pharmacologically acceptable alkali metal, alkaline earth metal or earth metal salts of weak acids, pharmacologically suitable hydroxides and oxides of alkaline earth and earth metals or else pharmacologically acceptable basic buffer systems. Examples which may be mentioned are sodium carbonate, calcium carbonate, magnesium carbonates, magnesium oxide, magnesium hydroxide, magnesium metasilicate aluminate, magnesium silicates, magnesium aluminate, hydrotalcite (synthetic), aluminium magnesium hydroxide, and calcium hydroxide, basic salts of amino adds, sodium hydroxide, trihydroxymethylaminomethane, trisodium citrate, disodium hydrogen phosphate and trisodium phosphate or mixtures thereof.

Preference is given according to the invention to sodium carbonate, disodium hydrogen phosphate, trisodium phosphate and buffer systems composed of disodium hydrogen phosphate with sodium hydroxide.

The basic excipient is preferably thoroughly mixed in finely divided form with the active ingredient and, where appropriate, other excipients or carriers so that there is intensive (direct) contact between basic excipient and the active ingredient. A further possibility is also to employ excipient granules impregnated with a basic buffer system.

The basic excipient is preferably added in an amount such that when 100 mg of mixtures of the active ingredient with the desired excipients are dissolved in 50 ml of purified water the basicity reaches not less than pH 7, preferably a basicity of pH 8 to pH 11.5, particularly preferably of pH 8 to pH 11.0 and very particularly preferably of pH 8.5 to 10.5. Depending on the nature of the basic excipient, the content can therefore be for example from 0.1 to 30% by weight (in percent by weight based on the finished dosage form). In a preferred embodiment the content of the basic excipient is below 20% by weight, particularly preferable below 15% by weight and in particular below 10% by weight (in percent by weight based on the finished dosage form).

Further excipients which can be used in the dosage forms according to the invention are, for example, excipients which bring about rapid disintegration of the dosage form on oral intake of the dosage form. These preferably comprise one or more substances selected from the group of fillers or carriers and disintegrants. It is furthermore possible for one or more excipients from the group of binders, lubricants, colouring agents, aromas, flavourings and surface-active substances to be present.

Fillers or carriers suitable according to the invention are, in particular, fillers such as calcium carbonate (e.g. MagGran® CC or Destab® 95) and sodium carbonate, sugar alcohols such as mannitol (e.g. Perlitol® or Parteck® M), sorbitol (e.g. Karion®), xylitol or maltitol, starches such as corn starch, potato starch and wheat starch, microcrystalline cellulose, saccharides such as glucose, lactose, levulose, sucrose and dextrose. Microcrystalline cellulose and/or mannitol are particularly preferred.

The content (in percent by weight based on the finished dosage form) of filler in the dosage form according to the invention is advantageously from 1 to 99% by weight. The content of filler is preferably from 30 to 95% by weight, and the content is particularly preferably from 60 to 90% by weight.

Disintegrants suitable according to the invention are, in particular, insoluble polyvinylpyrrolidone (Insoluble PVP, crosspovidone), sodium carboxymethyl starch, sodium carboxymethylcellulose, alginic acid, and starches able to fulfil the function of a disintegrant (e.g. Starch 1500).

The content in percent by weight based on the dosage form according to the invention) of disintegrant in the rapidly disintegrating dosage form according to the invention can usually be from 0.5 to 30% by weight. The content of disintegrant is preferably from 1 to 15% by weight. The content of disintegrant is particularly preferably from 1 to 5% by weight.

Suitable lubricants which may be mentioned are sodium stearyl fumarate, magnesium stearate, calcium stearate, stearic add, talc and colloidal silica (Aerosil).

The content (in percent by weight based on the finished dosage form) of lubricant in the rapidly disintegrating dosage form according to the invention is usually from 0.1 to 5% by weight. The content of lubricant is preferably from 0.2 to 3% by weight. The content of lubricant is particularly preferably from 0.5 to 2% by weight.

Binders suitable according to the invention are polyvinylpyrrolidone (PVP, Polyvidon® K25, Polyvidon® K90) or mixtures of PVP with polyvinyl acetate (e.g. Kollidon® 64), gelatin, corn starch paste, preswollen starches (Starch® 1500, Uni-Pure® WG220), hydroxypropylmethylcellulose (HPMC) or hydroxypropylcellulose (L-HPC).

The content on percent by weight based on the finished dosage form according to the invention) of binder can be up to 10% by weight, and it can preferably be up to 5% by weight.

Suitable surface-active substances which may be mentioned are sodium lauryl sulfate or Tween® 20, Tween® 60 or Tween® 80.

The dosage form according to the invention particularly preferably contains a mixture of at least one basic excipient, one filler or carrier, one disintegrant and one lubricant.

A dosage form which may be mentioned as preferred in this connection is one containing microcrystalline cellulose as filler or carrier and sodium carbonate as basic excipient and, in addition, a disintegrant and a lubricant. In another embodiment, the dosage form according to the invention contains a mixture of at least one basic excipient, one filler or carrier, one disintegrant, one binder and one lubricant. A dosage form which may be mentioned as preferred in this connection is one containing a mixture which contains mannitol and microcrystalline cellulose as filler or carrier, sodium carbonate as basic excipient and, in addition, binder and disintegrant. Another dosage form dosage form which may be mentioned as preferred in this connection is one containing a mixture which contains microcrystalline cellulose, sodium carbonate, sodium carboxymethyl starch and magnesium stearate.

It is also possible if desired for one or more flavourings (e.g. aromas or sweeteners) to be present in the dosage form according to the invention. It is possible thereby for example to achieve an improvement in taste. These substances are added in the usual amounts.

It is also possible if desired to use suitable colouring agents such as, for example, iron oxides, indigo-carmin E132 or titanium dioxide. These can either be processed directly in the mixture with the active ingredient to give coloured dosage forms, or be applied as ingredient of film coatings to the dosage forms.

Suitable for the film coating in the case of coated dosage forms according to the invention (such as, for example, coated tablets) are substances suitable for film coating. Examples which may be mentioned are cellulose esters such as hydroxypropylmethylcellulose (HPMC) and hydroxypropylcellulose (L-HPC), polyvinyl alcohol, phthalates and polymethacrylates (e.g. Eudragits®), to which plasticizers (such as, for example, propylene glycol, polyethylene glycols, trisodium citrate) and/or further additives and excipients (e.g. buffers, bases such as, preferably, aluminium hydroxide or pigments) can also be added if desired. In the case of film coatings, the content (in % by weight based on the finished dosage form) is from 1 to 20% by weight, preferably from 2 to 5% by weight in the case of dosage forms containing photosensitive reversible proton pump inhibitors it is preferred for a coloured film coating to be applied to the dosage forms according to the invention or for dyes to be incorporated directly into the dosage forms. Examples of film coatings which may be mentioned for the production of coloured dosage forms are OPADRY® (e.g. OPADRY® GREEN or OPADRY II® GREEN). OPADRY® GREEN comprises mixtures of hydroxypropylmethylcellulose/hypromellose, titanium dioxide, macrogol/PEG, yellow iron oxide and indigo-carmine E132 and OPADRY II® GREEN comprises mixtures of polyvinyl alcohol, titanium dioxide, macrogol/PEG, yellow iron oxide, black iron oxide and indigo E32.

In a preferred embodiment according to the invention the dosage form is comprising (7R,8R,9R)-2,3-dimethyl-8-hydroxy-7-(2-methoxyethoxy)-9-phenyl-7,8,9,10-tetrahydroimidazo[1,2-h][1,7]naphthyridine (INN soraprazan) or a pharmacologically acceptable salt and/or hydrate thereof as proton pump antagonist, sodium carbonate as basic excipient and microcrystalline cellulose, sodium carboxymethyl starch and magnesium stearate as excipients. In a further embodiment such dosage form is a film coated tablet. Particularly preferably such dosage form comprises a coloured film coating. Preferably the dosage form shows a disintegration time determined in water at 37° C. of not more than 5 min and dissolution (release of active ingredient) greater than or equal to 85% after 15 minutes in 0.1 N hydrochloric acid.

The dosage form according to the invention is produced by processes known to the skilled person, in particular by mixing the proton pump antagonists with the excipients. It is preferred in this connection for the active ingredient to be mixed thoroughly with the basic excipient in the case of tablets, the rapidly disintegrating dosage form is preferably produced by dry mixing of the excipients with the active ingredient. If desired, the active ingredient can be premixed with part of the filer or carrier. Conventional mixers such as compulsory mixers or free-fall mixers can be employed for the mixing operation. An alternative possibility is to produce granules of the ingredients of the dosage form and then compress them to tab lets. The preparations obtained in this way can then be compressed on a suitable tablet press. If desired, precompaction may also take place. In the case of coated tablets, the desired film coating is then applied in conventional ways using the equipment customary for these purposes (e.g. coating pans or drum coaters). Water is preferably used as granulating and coating liquid. In the case of coloured dosage forms the colouring agent is preferably dispersed homogeneously in the granules, or admixed dry, and then moistened or granulated or suspended in the dye pigment in the granulating liquid.

In the case of pellets, the rapidly disintegrating dosage form is preferably produced by spraying a basified active ingredient preparation onto starter pellets or by the extruder/spheronizer process.

### EXAMPLES

The following formulation examples illustrate the invention in detail without restricting it.

### EXAMPLES

### Example 1

Film-coated tablets:
I. Production of the uncoated core:

| | | |
|---|---|---|
| a) | soraprazan | 10.0 mg |
| b) | sodium carbonate (anhydrous) | 5.1 mg |
| c) | microcrystalline cellulose | 137.2 mg |
| | (e.g.: Avicel PH 102) | |
| d) | microcrystalline cellulose | 7.5 mg |
| | (e.g.: Avicel PH 101) | |
| e) | sodium carboxymethyl starch | 8.5 mg |
| f) | magnesium stearate | 1.7 mg |
| | | 170.0 mg |

a) is premixed with d) in a compulsory mixer. This mixture is admixed with b), c) and a) in the compulsory mixer. Subsequently f) is admixed in a free-fall mixer. The tableting mixture is compressed to cores in a suitable tablet press.

II. Film layer

| | |
|---|---|
| g) Opadry II green | 5.0 mg |
| | 175.0 mg |

g) is applied to the tablet cores obtained in 1. In a suitable film-coating apparatus.

### Example 2

Film-coated tablets:
I. Production of the uncoated core:

| | | |
|---|---|---|
| a) | soraprazan | 10.0 mg |
| b) | trisodium phosphate | 5.1 mg |
| c) | microcrystalline cellulose | 83.5 mg |
| | (e.g.: Avicel PH 101) | |
| d) | mannitol | 51.0 mg |
| e) | sodium carboxymethyl starch | 5.1 mg |
| f) | Starch 1500 | 13.6 mg |
| g) | magnesium stearate | 1.7 mg |
| | | 170.0 mg |

II. Film layer

| | | |
|---|---|---|
| h) | Opadry II green | 3.1 mg |
| | | 173.1 mg |

### Example 3

Film-coated tablets:
I. Production of the uncoated core:

| | | |
|---|---|---|
| a) | soraprazan | 20.0 mg |
| b) | sodium carbonate (anhydrous) | 10.2 mg |
| c) | microcrystalline cellulose | 114.6 mg |
| | (e.g.: Avicel PH 102) | |
| d) | microcrystalline cellulose | 15.0 mg |
| | (e.g.: Avicel PH 101) | |
| e) | Primojel | 8.5 mg |
| f) | magnesium stearate | 1.7 mg |
| | | 170.0 mg |

a) is premixed with d) in a compulsory mixer. This mixture is admixed with b), c) and e) in the compulsory mixer. Subsequently f) is admixed in a free-tall mixer. The tabletting mixture is compressed to cores in a suitable tablet press.

II. Film layer

| | |
|---|---|
| g) Opadry II green | 5.0 mg |
| | 175.0 mg |

g) is applied to the tablet cores obtained in 1. In a suitable film-coating apparatus.

### Example 4

Film-coated tablets:
I. Production of the uncoated core:

| | | |
|---|---|---|
| a) | soraprazan | 20.0 mg |
| b) | sodium carbonate (anhydrous) | 10.2 mg |
| c) | microcrystalline cellulose | 274.4 mg |
| | (e.g.: Avicel PH 102) | |
| d) | microcrystalline cellulose | 15.0 mg |
| | (e.g.: Avicel PH 101) | |
| e) | Primojel | 17.0 mg |
| f) | magnesium stearate | 3.4 mg |
| | | 340.0 mg |

a) is premixed with d) in a compulsory mixer. This mixture is admixed with b), c) and e) in the compulsory mixer. Subsequently f) is admixed briefly in a free-fall mixer. The tabletting mixture is compressed to cores in a suitable tablet press.

II. Film layer

| | |
|---|---|
| g) Opadry II green | 7.5 mg |
| | 347.5 mg |

g) is applied to the tablet cores obtained in I, in a suitable film-coating apparatus.

### Example 5

Film-coated tablets:
I. Production of the uncoated core:

| | | |
|---|---|---|
| a) | soraprazan | 20.0 mg |
| b) | sodium carbonate (anhydrous) | 5.1 mg |
| c) | microcrystalline cellulose | 119.7 mg |
| | (e.g.: Avicel PH 102) | |
| d) | microcrystalline cellulose | 15.0 mg |
| | (e.g.: Avicel PH 101) | |
| e) | Primojel | 8.5 mg |
| f) | magnesium stearate | 1.7 mg |
| | | 170.0 mg |

a) is premixed with d) in a compulsory mixer. This mixture is admixed with b), c) and e) in the compulsory mixer. Subsequently f) is admixed briefly in a free-fag mixer. The tabletting mixture is compressed to cores in a suitable tablet press.

II. Film layer

| | |
|---|---|
| g) Opadry II green | 5.0 mg |
| | 175.0 mg |

g) is applied to the tablet cores obtained in I. In a suitable film-coating apparatus.

### Example 6

Film-coated tablets:

| | | |
|---|---|---|
| a) soraprazan | 5.0 | mg |
| b) mannitol | 50.0 | mg |
| c) microcrystalline cellulose (e.g.: Avicel PH 101) | 20.0 | mg |
| d) Uni Pure ® WG 220 | 3.0 | mg |
| e) basic buffer | 2.0 | mg |
| Mass of granules | 80.0 | mg |
| f) disintegrant | 4.0 | mg |
| g) magnesium stearate | 0.25 | mg |
| Mass of tablet core | 84.25 | mg |
| h) film coating | 4.0 | mg |
| Mass of film-coated tablet | 88.25 | mg |

### Example 7

Film-coated tablets:

| | | |
|---|---|---|
| a) soraprazan | 5.0 | mg |
| b) mannitol | 50.0 | mg |
| c) microcrystalline cellulose | 20.0 | mg |
| (e.g.: Avicel PH 101) | | |
| d) Uni Pure ® WG 220 | 3.0 | mg |
| e) basic buffer | 2.0 | mg |
| Mass of granules | 80.0 | mg |
| f) disintegrant | 4.0 | mg |
| g) magnesium stearate | 0.25 | mg |
| Mass of table core | 84.25 | mg |
| h) film coating | 4.0 | mg |
| Mass of film-coated tablet | 88.25 | mg |

### Example 8

Film-coated tablets:

| | | |
|---|---|---|
| a) soraprazan | 5.0 | mg |
| b) mannitol | 50.0 | mg |
| c) microcrystalline cellulose | 20.0 | mg |
| (e.g.: Avicel PH 101) | | |
| d) Uni Pure ® WG 220 | 3.0 | mg |
| e) sodium carbonate | 1.2 | mg |
| Mass of granules | 79.2 | mg |
| f) Explotab | 4.0 | mg |
| g) magnesium stearate | 0.25 | mg |
| Mass of table core | 83.45 | mg |
| h) film coating (PVA-based) | 3.55 | mg |
| Mass of film-coated tablet | 87.00 | mg |

### Example 9

Film-coated tablets:

| | | |
|---|---|---|
| a) soraprazan | 20.0 | mg |
| b) mannitol | 124.0 | mg |
| c) microcrystalline cellulose | 52.0 | mg |
| (e.g.: Avicel PH 101) | | |
| d) Uni Pure ® WG 220 | 8.2 | mg |
| e) sodium carbonate | 3.3 | mg |
| Mass of granules | 207.5 | mg |
| f) Explotab | 11.0 | mg |
| g) magnesium stearate | 0.7 | mg |
| Mass of tablet core | 219.2 | mg |
| h) film coating (PVA-based) | 9.8 | mg |
| Mass of film tablets | 229.00 | mg |

| | Example 10 | | Example 11 | | Example 12 | | Example 13 | |
|---|---|---|---|---|---|---|---|---|
| soraprazan | 5.00 | mg | 5.00 | mg | 5.00 | mg | 5.00 | mg |
| sodium carbonate, anhydrous | | | | | | | | |
| disodium hydrogen phosphate | 2.40 | mg | 2.40 | mg | 2.40 | mg | 2.40 | mg |
| microcrystalline cellulose in | 20.00 | mg | 20.00 | mg | 13.00 | mg | 13.00 | mg |
| the form of Avicel PH 101 | | | | | | | | |
| microcrystalline cellulose in | | | | | | | | |
| the form of Avicel PH 112 | | | | | | | | |
| mannitol | 49.27 | mg | 49.27 | mg | 31.00 | mg | 31.00 | mg |
| Ac-Di-Sol | 4.25 | mg | 4.25 | mg | 2.75 | mg | 2.75 | mg |
| Uni-Pure WG 220 | 1.60 | mg | 1.60 | mg | 1.60 | mg | 1.60 | mg |
| indigocarmine E132 | 0.43 | mg | 0.43 | mg | | | | |
| yellow iron oxide | 0.30 | mg | 0.30 | mg | | | | |
| magnesium stearate | 0.25 | mg | 0.25 | mg | 0.25 | mg | 0.25 | mg |
| subcoat PVA clear | | | | | | | | |
| subcoat HPMC clear | | | | | | | | |
| coating HPMC/FeO | | | | | 3.00 | mg | 3.00 | mg |
| coating PVA/FeO | | | | | | | | |
| coating | | | | | | | | |
| PVA/FeO/indigocarmine | | | | | | | | |
| E132 | | | | | | | | |
| Total for film-coated tablet | 83.50 | mg | 83.50 | mg | 59.00 | mg | 59.00 | mg |
| | Example 14 | | Example 15 | | Example 16 | | Example 17 | |
| soraprazan | 5.00 | mg | 5.00 | mg | 5.00 | mg | 5.00 | mg |
| sodium carbonate, anhydrous | | | | | 1.20 | mg | 1.20 | mg |
| disodium hydrogen phosphate | 2.40 | mg | 2.40 | mg | | | | |
| microcrystalline cellulose in | 20.00 | mg | 20.00 | mg | 20.00 | mg | 20.00 | mg |
| the form of Avicel PH 101 | | | | | | | | |
| microcrystalline cellulose in | | | | | | | | |
| the form of Avicel PH 112 | | | | | | | | |
| mannitol | 50.00 | mg | 50.00 | mg | 50.00 | mg | 50.00 | mg |
| Ac-Di-Sol | 4.25 | mg | 4.25 | mg | 3.95 | mg | 3.95 | mg |
| Uni-Pure WG 220 | 1.60 | mg | 1.60 | mg | 1.60 | mg | 1.60 | mg |
| indigocarmine E132 | | | | | | | | |
| yellow iron oxide | | | | | | | | |
| magnesium stearate | 0.25 | mg | 0.25 | mg | 0.25 | mg | 0.25 | mg |
| subcoat PVA clear | | | 0.855 | mg | | | | |
| subcoat HPMC clear | 0.855 | mg | | | | | | |
| coating HPMC/FeO | | | | | | | 4.10 | mg |
| coating PVA/FeO | | | | | 4.10 | mg | | |
| Coating | | | | | | | | |
| PVA/FeO/indigocarmine | | | | | | | | |
| E132 | 4.47 | mg | 4.47 | mg | | | | |
| Total for film-coated tablet | 88.83 | mg | 88.83 | mg | 86.10 | mg | 86.10 | mg |

| | Example 18 | | Example 19 | | Example 20 | |
|---|---|---|---|---|---|---|
| Core: | | | | | | |
| soraprazan | 10.0 | mg | 10.0 | mg | 10.0 | mg |
| sodium carbonate, anhydrous | 5.1 | mg | 5.1 | mg | 5.1 | mg |
| trisodium phosphate | | | | | | |
| trisodium phosphate | | | | | | |
| microcrystalline cellulose in | 137.2 | mg | 137.2 | mg | | |
| the form of Avicel PH 102 | | | | | | |
| microcrystalline cellulose in | 7.5 | mg | 7.5 | mg | 7.5 | mg |
| the form of Avicel PH 101 | | | | | | |
| microcrystalline cellulose in | | | | | 137.2 | mg |
| the form of Avicel PH 112 | | | | | | |
| mannitol | | | | | | |
| sodium carboxymethyl | 8.5 | mg | 8.5 | mg | 8.5 | mg |
| starch | | | | | | |
| pregelatinized starch (Starch | | | | | | |
| 1500) | | | | | | |
| magnesium stearate | 1.7 | mg | 1.7 | mg | 1.7 | mg |
| Total for core | 170.0 | mg | 170.0 | mg | 170.0 | mg |
| Film: | | | | | | |
| Opadry green 03F21409 | 5.0 | mg | | | 5.0 | mg |
| Opadry II green 85F21399 | | | 5.0 | mg | | |
| Total for film-coated tablet | 175.0 | mg | 175.0 | mg | 175.0 | mg |
| | Example 21 | | Example 22 | | Example 23 | |
| Core: | | | | | | |
| soraprazan | 10.0 | mg | 10.0 | mg | 10.0 | mg |
| sodium carbonate, anhydrous | 5.1 | mg | | | | |
| trisodium phosphate | | | 5.1 | mg | 2.89 | mg |
| trisodium phosphate | | | | | 2.21 | mg |
| microcrystalline cellulose in | | | | | | |
| the form of Avicel PH 102 | | | | | | |
| microcrystalline cellulose in | 7.5 | mg | 83.5 | mg | 83.5 | mg |
| the form of Avicel PH 101 | | | | | | |
| microcrystalline cellulose in | 137.2 | mg | | | | |
| the form of Avicel PH 112 | | | | | | |
| mannitol | | | 51 | mg | 51 | mg |
| sodium carboxymethyl | 8.5 | mg | 5.1 | mg | 5.1 | mg |
| starch | | | | | | |
| pregelatinized starch (Starch | | | 13.6 | mg | 13.6 | mg |
| 1500) | | | | | | |
| magnesium stearate | 1.7 | mg | 1.7 | mg | 1.7 | mg |
| Total for core | 170.0 | mg | 170.0 | mg | 170.0 | mg |
| Film: | | | | | | |
| Opadry green 03F21409 | | | | | 4.4 | mg |
| Opadry II green 85F21399 | 5.0 | mg | 3.1 | mg | | |
| Total for film-coated tablet | 175.0 | mg | 173.1 | mg | 174.4 | mg |
| | Example 24 | | Example 25 | | Example 26 | |
| Core: | | | | | | |
| soraprazan | 10.0 | mg | 10.0 | mg | 10.0 | mg |
| sodium carbonate, anhydrous | | | | | 5.1 | mg |
| trisodium phosphate | 5.1 | mg | 2.89 | mg | | |
| trisodium phosphate | | | 2.21 | mg | | |
| microcrystalline cellulose in | 83.5 | mg | 83.5 | mg | 83.5 | mg |
| the form af Avicel PH 102 | | | | | | |
| microcrystalline cellulose in | | | | | | |
| the form of Avicel PH 101 | | | | | | |
| microcrystalline cellulose in | | | | | | |
| the form of Avicel PH 112 | | | | | | |
| mannitol | 51 | mg | 51 | mg | 51.0 | mg |
| sodium carboxymethyl | 5.1 | mg | 5.1 | mg | 5.1 | mg |
| starch | | | | | | |
| pregelatinized starch (Starch | 13.6 | mg | 13.6 | mg | 13.6 | mg |
| 1500) | | | | | | |
| magnesium stearate | 1.7 | mg | 1.7 | mg | 1.7 | mg |
| Total for core | 170.0 | mg | 170.0 | mg | 170.0 | mg |
| Film: | | | | | | |
| Opadry green 03F21409 | 4.8 | mg | | | 5.0 | mg |
| Opadry II green 85F21399 | | | 3.3 | mg | | |
| Total for film-coated tablet | 174.8 | mg | 173.3 | mg | 175.0 | mg |
| | Example 27 | | Example 28 | | Example 29 | |
| Core: | | | | | | |
| soraprazan | 10.0 | mg | 10.0 | mg | 10.0 | mg |
| sodium carbonate, anhydrous | 5.1 | mg | 5.1 | mg | 5.1 | mg |
| trisodium phosphate | | | | | | |
| trisodium phosphate | | | | | | |
| microcrystalline cellulose in | 83.5 | mg | | | | |
| the form of Avicel PH 102 | | | | | | |
| microcrystalline cellulose in | | | | | | |
| the form of Avicel PH 101 | | | | | | |
| microcrystalline cellulose in | | | 83.5 | mg | 83.5 | mg |
| the form of Avicel PH 112 | | | | | | |
| mannitol | 51.0 | mg | 51.0 | mg | 51.0 | mg |
| sodium carboxymethyl | 5.1 | mg | 5.1 | mg | 5.1 | mg |
| starch | | | | | | |
| pregelatinized starch (Starch | 13.6 | mg | 13.6 | mg | 13.6 | mg |
| 1500) | | | | | | |
| magnesium stearate | 1.7 | mg | 1.7 | mg | 1.7 | mg |
| Total for core | 170.0 | mg | 170.0 | mg | 170.0 | mg |

| Film: | NGa 5 | | NGa 8 | | NGa 9 | |
|---|---|---|---|---|---|---|
| Opadry green 03F21409 | | | 5.0 | mg | | |
| Opadry II green 85F21399 | 5.0 | mg | | | 5.0 | mg |
| Total for film-coated tablet | 175.0 | mg | 175.0 | mg | 175.0 | mg |

wet-granulated ingredients are shown in italics

Determination of the Disintegration Time for Tablet of Example 1

A film-coated tablet is subjected to a disintegration test under the test conditions described in the European Pharmacopoeia 4^{th} edition for “Dispersible Tablets”. The tablet is observed to disintegrate within 3 minutes in water at 15 to 25° C. A dispersion forms and can be poured through the screen (710).

Stability Testing

Triturations of soraprazan with different excipients including or excluding a basic excipient were manufactured, stored at 50° C. and analysed for impurities. The following results were obtained:

| | | | | Soraprazan, | Soraprazan, | Soraprazan, |
|---|---|---|---|---|---|---|
| | | | | Corn Starch, | Corn Starch, | Mannit |
| | | | | Mannit, | Magnesium | Magnesium |
| | | | | Magnesium | Stearate, | Stearate, |
| | | Soraprazan, | | Stearate, | Sodium | Sodium |
| | Soraprazan, | Magnesium | Soraprazan, | Disodium | Hydrogen- | Hydrogen- |
| Mixture | Mannit | Stearate | Corn Starch | Carbonate | carbonate | carbonate |
| Impurities | 5.29 | 5.01 | 6.67 | 3.76 | 3.68 | 3.74 |
| total | | | | | | |
| (AU %) | | | | | | |

For triturations comprising a basic exipient a distinct lower impurity profile is observed.

### INDUSTRIAL APPLICABILITY

Proton pump antagonists and their salts have valuable pharmacological properties which make them industrially utilizable. They show in particular a pronounced inhibition of gastric acid secretion and an excellent gastrointestinal-protective effect in warm-blooded species, especially humans. The compounds according to the invention are distinguished in this connection by a high selectivity of effect, an advantageous duration of action, a particularly good enteral activity, the absence of substantial side effects and a high therapeutic index.

“Gastrointestinal protection” means in this connection the prevention and treatment of gastrointestinal disorders, especially gastrointestinal inflammatory disorders and lesions (such as, for example, gastric ulcer, duodenal ulcer, gastritis, hyperacidic or drug-related dyspepsia, heartburn and acid eructation, severe reflux oesophagitis, prophylaxis of recurrent reflux oesophagitis and of duodenal ulcer, reflux oesophagitis, Zollinger-Ellison syndrome, elimination of the pathogen Helicobacter pylori in combination with amoxicillin and clarithromycin or in combination with clarithromycin and metronidazole or with amoxicillin and metronidazole, long-term treatment for prophylaxis of recurrent severe forms of reflux oesophagitis. Prophylaxis and therapy of ulcers and gastroduodenal erosions induced by non-steroidal antiinflammatory drugs), which may be caused for example by microorganisms (e.g. Helicobacter pylori), bacteriotoxins, medicines (e.g. certain antiinflammatory and antirheumatic drugs), chemicals (e.g. ethanol), gastric acid or stress situations.

Owing to these properties, the dosage forms according to the invention containing a proton pump antagonist and/or a pharmacologically acceptable salt thereof are outstandingly suitable for use in human and veterinary medicine, being used in particular for the treatment and/or prophylaxis of disorders of the stomach and/or intestine.

The invention therefore further relates to the dosage forms according to the invention for use in the treatment and/or prophylaxis of the aforementioned disorders.

The invention also includes the use of the dosage forms according to the invention for the treatment and/or prophylaxis of the aforementioned disorders. The dosage forms according to the invention may in this case be employed as such (e.g. direct oral intake by the patent) or be dissolved or dispersed in water before use. Particularly suitable for this purpose are the rapidly disintegrating dosage forms according to the invention which comply with the criteria of the European Pharmacopoeia 4^{th} edition (“Tablet for preparing a solution to be taken” or “Tablet for preparing a suspension to be taken”). The solutions or suspensions obtained after dispersion in a suitable dispersant or solvent can then be taken by the patient. This may, for example, be advantageous for patents who have problems with taking a solid dosage form. A further possibility is to administer such solutions or suspensions also by means of tubes (e.g. nose tubes, stomach tube). This is advantageous in particular on administration of the dosage forms according to the invention in patients receiving intensive care, patients with swallowing difficulties, bedridden patients and children.

The dosage forms according to the invention can be combined with other medicaments, either in different combinations or in a fixed combination. Combinations worth mentioning in connection with the dosage forms according to the invention containing proton pump antagonists as active ingredients are those with antimicrobial active ingredients and those with NSAIDs (non steroidal anti inflammatory drugs). Particular mention should be made of the combination with antimicrobial agents like those employed to control the microbe Helicobacter pylori (H. pylori). Further examples which may be mentioned of combinations are: tranquilizers (for example from the group of benzodiazepines, e.g. diazepam), spasmolytics (e.g. bi-etamiverine or camylofin), anticholinergics (e.g. oxyphencyclimine or phencarbamide), local anesthetics (e.g. tetracaine or procaine), where appropriate also enzymes, vitamins or amino acids. Combinations of the compounds according to the invention with drugs which inhibit acid secretion should be particularly emphasized in this connection, such as, for example, antacids, H2 blockers (e.g. cimetidine, ranitidine), H+/K+-ATPase inhibitors (e.g. omeprazole, pantoprazole), or else with so-called peripheral anticholinergics (e.g. pirenzepine, telenzepine) and with gastrin antagonists with the aim of enhancing the main effect in an additive or superadditive sense and/or of eliminating or reducing the side effects.

Examples of suitable antimicrobial active ingredients (active against Helicobacter pylori) are described in EP-A-0 282 131. Examples which may be mentioned of antimicrobial agents suitable for controlling the microbe Helicobacter pylori are for example bismuth salts [e.g. bismuth subcitrate, bismuth subsalicylate, ammonium bismuth(III) potassium citrate dihydroxide, bismuth nitrate oxide, dibismuth tris(tetraoxodialuminate)], especially β-lactam antibiotics, for example penicillins (such as benzylpenicillin, phenoxymethylpenicillin, propicillin, azidocillin, dicloxacillin, flucloxacillin, oxacillin, amoxicillin, bacampicillin, ampicillin, meziocillin, piperacillin or aziocillin), cephalosporins (such as cefadroxil, cefaclor, cefalexin, ceflixime, cefuroxime, cefetamet, cefadroxil, cettibuten, cefodoxime, cefotetan, cefazolin, cefoperazone, ceftizoxime, cefotaxime, ceftazidime, cefamandole, cefepime, cefoxitin, cefodizime, cefsulodin, ceftriaxone, cefotiam or cefmenoxine) or other β-lactam antibiotics (e.g. aztreonam, loracarbef or meropenem); enzyme inhibitors, for example sulbactam; tetracyclines, for example tetracycline, oxytetracycline, minocycline or doxycycline; aminoglycosides, for example tobramycin, gentamicin, neomycin, streptomycin, amikacin, netilmicin, paromomycin or spectinomycin; amphenicols, for example chloramphenicol or thiamphenicol; lincomycins and macrolide antibiotics, for example clindamycin, lincomycin, erythromycin, clarithromycin, spiramycin, roxithromycin or azithromycin; polypeptide antibiotics, for example colistin, polymixin B, telcoplanin or vancomycin; gyrase inhibitors, for example norfloxacin, cinoxacin, clprofloxacin, pipemidic acid, enoxacin, nalidixic acid, pefloxacin, fleroxacin or ofloxacin; nitroimidazoles, for example metronidazole; or other antibiotics, for example fosfomycin or fusidic acid. Administration of a reversible proton pump inhibitor together with the combination of a plurality of antimicrobial active ingredients is particularly worthy of mention in this connection, for example with the combination of a bismuth salt and/or tetracycline with metronidazole or the combination of amoxicillin or clarithromycin with metronidazole and amoxicillin with clarithromycin.

The dosage of the active ingredients in the dosage form according to the invention depends greatly on the nature of the proton pump antagonists used. A typical dosage for a proton pump antagonist as disclosed for example in WO-A-9418199 can be regarded as a daily dose of about 0.01 to about 20, preferably about 0.05 to 5, in particular 0.1 to 1.5, mg/kg of body weight, where appropriate in the form of a plurality of single doses. In the case of the compound soraprazan, examples of dosage forms according to the invention contain the proton pump antagonist in a dose of 2, 2.5, 5, 10, 15, 20 or 40 mg.

Antimicrobial active ingredients which may be emphasized are erythromycin, azithromycin, clarithromycin, clindamycin, rifampicin, ampicillin, meziocillin, amoxicillin, tetracycline, minocycline, doxycycline, imipenem, meropenem, cefalexin, cefuroxime axetil, cetpodoxime proxetil, cefaclor, cefadroxil, ciprofloxacin, norfloxacin, ofloxacin and pefloxacin.

Antimicrobial active ingredients which may be particularly emphasized are clarithromycin and amoxicillin.

Combined administration for the purposes of the present invention mean fixed and, in particular, free combination, i.e. either the proton pump antagonist and the antimicrobial active ingredient are present here in one dosage unit, or the proton pump antagonist and antimicrobial active ingredient, which are present in separate dosage units, are administered in direct succession or at a relatively large interval in time, a relatively large interval in time meaning a time span not exceeding 24 hours. For use as separate dosage units, these are preferably provided in a common package. For example, the two dosage units are packaged together in blisters which are designed in respect of the relative disposition of the two dosage units, the labelling and/or colouring in a manner known per se so that the time that the individual components (dosage regimen) of the two dosage units should be taken are evident to the patient.

Dosage unit means, in particular, dosage forms such as tablets, coated tablets or pellets, and microtablets in capsules, the dosage form advantageously being designed so that the two active ingredient components (proton pump antagonist on the one hand and antimicrobial active ingredient on the other hand) are released or effectively made available to the body in such a way that an optimal active ingredient profile and thus profile of effect is achieved.

1. An oral dosage form for proton pump antagonists (APA) comprising an effective amount of a proton pump antagonist, or a pharmacologically acceptable solvate, hydrate or salt thereof, together with one or more excipients, where the proton pump antagonist or the pharmacologically acceptable solvate, hydrate or salt thereof is stabilized in the dosage form by one or more basic excipients.

2. The dosage form according to claim 1, wherein the basic excipient is present in finely divided form and thoroughly mixed with the proton pump antagonist, or the pharmacologically acceptable solvate, hydrate or salt thereof.

3. The dosage form according to claim 1, characterized in that one or more excipients which, on oral intake of the dosage form, bring about rapid disintegration of the dosage form are present.

4. The dosage form according to claim 1, characterized in that the dosage form is selected from the group consisting of tablets, coated tablets, pellets, microtablets in capsules and granules in capsules.

5. The dosage form according to claim 4, characterized in that it comprises coated tablets.

6. The dosage form according to claim 3, characterized in that it comprises a rapidly disintegrating dosage form with immediate release of the active ingredient.

7. The dosage form according to claim 3, characterized in that it comprises a rapidly disintegrating dosage form with immediate release of the active ingredient, and wherein the dosage form shows a disintegration of not more than 5 minutes under the test conditions described for “Dispersible Tablets” in the European Pharmacopoeia 4^{th} edition.

8. The dosage form according to claim 3, characterized in that it comprises a rapidly disintegrating dosage form with immediate release of the active ingredient, and wherein the dosage form shows a disintegration within 3 minutes under the test conditions described for “Dispersible Tablets” in the European Pharmacopoeia 4^{th} edition.

9. The dosage form according to claim 7, characterized that it shows a release of the active ingredient of greater than or equal to 85% after 15 minutes in 0.1 N hydrochloric acid.

10. The dosage form according to claim 3, characterized in that one or more substances selected from the group consisting of fillers and disintegrants are present as excipients which bring about rapid disintegration of the tablet.

11. The dosage form according to claim 10, characterized in that at least one filler and at least one disintegrant are present.

12. The dosage form according to claim 11, characterized in that microcrystalline cellulose is present.

13. The dosage form according to claim 1, characterized in that one or more further excipients selected from the group consisting of lubricants, aromas, coloring agents, flavorings and surface-active substances are present.

14. The dosage form according to claim 1, characterized in that the basic excipient is selected from the group consisting of sodium carbonate, calcium carbonate, magnesium carbonates, magnesium oxide, magnesium hydroxide, magnesium metasilicate aluminate, magnesium silicates, magnesium aluminate, synthetic hydrotalcite, aluminium magnesium hydroxide, calcium hydroxide, basic salts of amino acids, sodium hydroxide, trihydroxymethylaminomethane, trisodium citrate, disodium hydrogen phosphate, trisodium phosphate and mixtures thereof.

15. The dosage form according to claim 14, characterized in that the basic excipient is sodium carbonate is.

16. The dosage form according to claim 14, characterized in that the basic excipient is disodium hydrogen phosphate, trisodium phosphate or a buffer of system comprising disodium hydrogen phosphate and sodium hydroxide.

17. The dosage form according to claim 1, wherein the proton pump antagonist (APA) is selected from the group consisting of AU-461, soraprazan (BYK61359), DBM-819, KR-60436, T-330, YH-1885, YJA-20379-8 and 2,3-dimethyl-8-(2-ethyl-6-methylbenzylamino)imidazo[1,2-a]pyridine-6-carboxamide.

18. The dosage form according to claim 17, characterized in that (7R,8R,9R)-2,3-dimethyl-8-hydroxy-7-(2-methoxyethoxy)-9-phenyl-7,8,9,10-tetrahydroimidazo[1,2-h][1,7]naphthyridine (INN soraprazan) or a pharmacologically acceptable solvate, hydrate or salt thereof is present as the proton pump antagonist.

19. The dosage form according to claim 9, comprising (7R,8R,9R)-2,3-dimethyl-8-hydroxy-7-(2-methoxyethoxy)-9-phenyl-7,8,9,10-tetrahydroimidazo[1,2-h][1,7]naphthyridine (INN soraprazan) or a pharmacologically acceptable solvate, hydrate or salt thereof as the proton pump antagonist, sodium carbonate as the basic exipient and microcrystalline cellulose, sodium carboxymethyl starch and magnesium stearate as the exipients.

20. The dosage form according to claim 19, which is a film coated tablet.

21. The dosage form according to claim 20, which comprises a colored film coating.

22. The method for preparing a dosage form according to claim 1 comprising the step of thoroughly mixing the active ingredient with the basic excipient.

23. A rapidly disintegrating dosage form comprising an effective amount of a proton pump antagonist (APA), or a pharmacologically acceptable solvate, hydrate or salt thereof, together with one or more excipients which, on oral intake of the dosage form, brings about rapid disintegration of the dosage form.

24. The dosage form according to claim 23, which dosage form shows an immediate release of the proton pump antagonist (APA).

25. The dosage form according to claim 24, which shows a disintegration time determined in water at 37° C. of not more than 5 min and a release of active ingredient greater than or equal to 85% after 15 minutes in 0.1 N hydrochloric acid.

26. The dosage form according to claim 23, characterized in that (7R,8R,9R)-2,3-dimethyl-8-hydroxy-7-(2-methoxyethoxy)-9-phenyl-7,8,9,10-tetrahydroimidazo[1,2-h][1,7]naphthyridine (INN soraprazan) or a pharmacologically acceptable solvate, hydrate or salt thereof is present as the proton pump antagonist.
